(19) United States
(12) Patent Application Publication
(10) Pub. No.: US 2018/0110990 A1
(43) Pub. Date: Apr. 26, 2018

(54) SYSTEM AND METHOD USING GESTURES TO CONTROL ELECTROTHERAPY DELIVERY FROM AN ELECTROTHERAPY DEVICE

(71) Applicant: BIOTRONIK SE & CO. KG, Berlin (DE)

(72) Inventors: RAINER JOERG GROSSKOPF, PORTLAND, OR (US); MICHAEL REINERT, TIGARD, OR (US)

(21) Appl. No.: 15/716,562

(22) Filed: Sep. 27, 2017

Related U.S. Application Data
(60) Provisional application No. 62/410,440, filed on Oct. 20, 2016.

(51) Int. Cl. A61N 1/36; A61N 1/372; G06F 3/01

(52) U.S. Cl. CPC A61N 1/36128; G06F 3/04883; G06F 3/016; A61N 1/37247

(57) ABSTRACT

A system for wireless control of an implantable stimulation device contains an implantable stimulation device for delivering electrical stimulation to a patient and an external control device. The external control device is configured to transmit a control signal to the stimulation device when the external control device detects a change in translational and/or rotational motion, or a contactless interaction of the patient with the external control device.

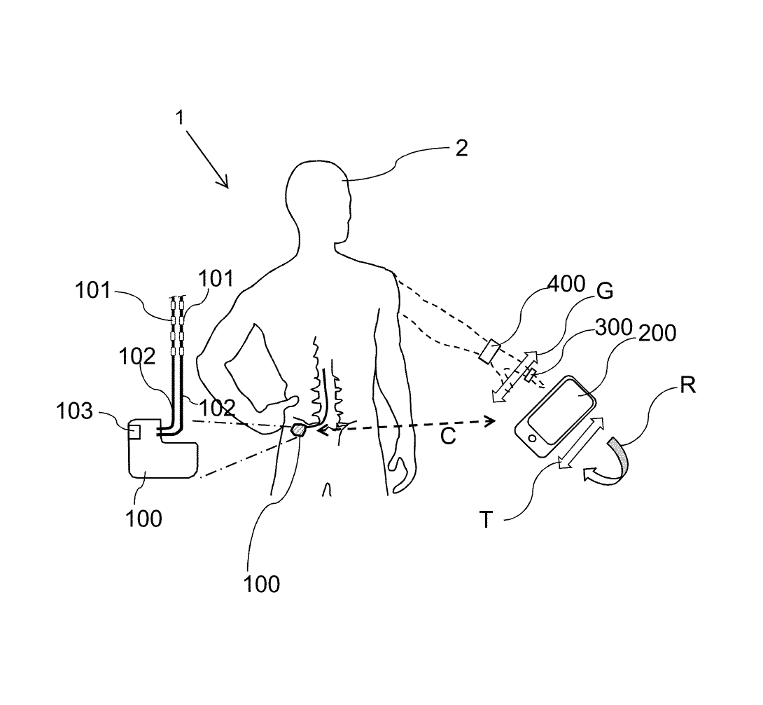

### CROSS-REFERENCE TO RELATED APPLICATION

This application claims the priority, under 35 U.S.C. § 119(e), of provisional patent application No. 62/410,440 filed Oct. 20, 2016; the prior application is herewith incorporated by reference in its entirety.

### BACKGROUND OF THE INVENTION

### Field of the Invention

The present invention relates to a system and method using gestures to control electrotherapy delivery from an electrotherapy device, particularly for providing spinal cord stimulation (SCS).

Spinal cord stimulators provide electrotherapy to reduce pain in patients. Patients frequently adjust the therapy or stimulation parameters in response to their current activities (e.g. sitting, standing, driving, walking, sleeping etc.).

U.S. patent publication No. 2011/0270358 describes programming a spinal cord stimulation system using a gesture-based control done by a physician during programming of parameter settings for the patient. Further, international patent disclosure WO 2013/011483 A2 (corresponding to U.S. Pat. No. 9,579,510) describes a remote control for a medical device, particularly a hearing aid, based on tags (e.g. radio frequency identification (RFID) tags).

### SUMMARY OF THE INVENTION

Based on the above, the problem addressed by the present invention is to provide a system and a method of the afore-mentioned kind having an improved handling.

The problem is solved by a system having the features of the main claim. Preferred embodiments of these aspects of the present invention are stated in the corresponding sub-claims and are described below.

According to the main claim, a system for wireless control of an implantable stimulation device is disclosed. The system contains an implantable stimulation device for delivering electrical stimulation to a patient and an external control device. The external control device is configured to transmit a control signal to the stimulation device when the external control device detects:
a) a change in translational and/or rotational motion, or
b) a contactless interaction of the patient with the external control device.

Particularly the stimulation device is formed as an implantable pulse generator (IPG).

Further, according to an embodiment of the system according to the invention, the stimulation device is configured for spinal cord stimulation. The stimulation device contains a plurality of electrodes and the stimulation device is configured to deliver spinal cord stimulation via the electrodes.

According to an embodiment of the present invention, the stimulation device may contain one or even two elongated flexible carriers. Each of the carriers contains a plurality of electrodes at a distal portion via which SCS may be applied to the spinal cord. Particularly, electrodes of one carrier may act as anodes wherein electrodes of the other carrier may act as cathodes. The two carriers particularly extend along one another.

Further, according to an embodiment of the system according to the invention, the contactless interaction with the external control device (e.g. smartphone) is a motion gesture of the patient in front of the external control device which in this case is particularly configured to detect the gesture e.g. by use of a camera integrated into the external control device or by use of a further sensor connected to the hand that communicates with the external control device.

Particularly, according to an embodiment of the present invention, the external control device is one of or contains at least one of: a hand-held device, a smartphone, a smartwatch, an object configured to be worn by the patient, a bracelet, a ring.

Particularly, in an embodiment, the external control device (e.g. smartphone), has a software installed that provides a control interface to the stimulation device via a radio connection (e.g. Bluetooth). The external control device further has a magnetic field sensor that senses the orientation of the external control device relative to the earth's magnetic field such that when the external control device is rotated by the patient (e.g. when lying on a surface) a corresponding rotation angle is translated into an increase or a decrease of a parameter (e.g. stimulation amplitude) of the electrical stimulation of the stimulation device depending on the direction of the rotation (i.e. the external control device can be used like a rotary knob).

Further, in an embodiment, the system further contains one or several rings configured to be arranged on fingers of the patient, respectively, which ring(s) are configured to be connected to the external control device (e.g. smart phone) via a radio connection (e.g. Bluetooth). The respective ring contains a motion sensor, and the external control device (e.g. smartphone) has a software installed that acts as control interface to the stimulation device via a radio connection (e.g. Bluetooth) such that when the patient performs (e.g. taps) a specific finger pattern or movement that is detected by the ring(s), a parameter of the electrical stimulation generated by the stimulation device is changed accordingly.

Furthermore, in an embodiment, the external control device (e.g. smartphone), has a software installed that provides a control interface to the stimulation device via a radio connection (e.g. Bluetooth). The external control device (e.g. smartphone) further contains a camera that senses a specific movement of the patient (e.g. of its hand) that is translated to a corresponding adjustment of a parameter (e.g. stimulation amplitude) of the electrical stimulation of the stimulation device.

Furthermore, in an embodiment, the external control device is formed as a smartwatch that has a software installed that provides a control interface to the stimulation device via a radio connection (e.g. Bluetooth). The smartwatch contains a motion sensor that senses a specific movement of the patient (e.g. of its wrist) that is translated to a corresponding adjustment of a parameter (e.g. stimulation amplitude) of the electrical stimulation of the stimulation device.

Particularly, in the framework of the present invention, a smartphone is a cell phone that contains a display, particularly in the form of a touch-screen, and a microprocessor, and is capable of executing software applications that are installed in a memory on the phone. Likewise, particularly, a smartwatch is a watch that contains a display, particularly in the form of a touch-screen, and a microprocessor, and is capable of executing software applications that are installed in a memory on the watch. Moreover, the smartphone may maintain a direct or indirect wireless communication link to the stimulation device.

Particularly, instead of a smartwatch, also a bracelet can be used which then contains the motion sensor configured to detect complex finger gestures or wrist movements.

Furthermore, the external control device can also be formed by one or several pressure-sensitive finger rings that are configured to detect gestures of the hand of the patient wearing said ring(s).

Further, according to an embodiment of the system according to the invention, one of the following parameters of the electrical stimulation is adjusted by the external control device:
a) stimulation amplitude,
b) stimulation pulse width,
c) stimulation frequency and/or stimulation cycles, and
d) electrode or electrodes for stimulation from a plurality of electrodes, e.g. selection of one or more electrodes from an array of electrodes.

A further aspect of the present invention relates to a method for wireless control of an implantable stimulation device using an external control device, wherein

a change in translational and/or rotational motion of the external control device, or a contactless interaction of the patient with the external control device is detected by the external control device, and a corresponding control signal is transmitted by the external control device to the stimulation device for controlling the stimulation device.

Particularly, the method according to the invention uses a system according to the present invention as described herein.

Particularly, according to an embodiment of the method according to the present invention, the contactless interaction with the external control device is a motion gesture of the patient in front of the external control device.

Particularly, according to an embodiment of the method according to the present invention, one of the following parameters is adjusted by of the external control device:
a) stimulation amplitude,
b) stimulation pulse width,
c) stimulation frequency and/or stimulation cycles, and
d) electrode or electrodes for stimulation from a plurality of electrodes, e.g. selection of one or more electrodes from an array of electrodes.

Furthermore, particularly, the radio communication between the external control device and the stimulation device and between other component such as ring (see above) and the external control device are particularly conducted in the range from 2,402 GHz to 2,480 GHz (e.g. using Bluetooth).

Other features which are considered as characteristic for the invention are set forth in the appended claims.

Although the invention is illustrated and described herein as embodied in a system and a method using gestures to control electrotherapy delivery from an electrotherapy device, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWING

The single FIGURE of the drawing is a schematic representation of a system according to the present invention for wireless control of a electrotherapy device, particularly of a system for spinal cord stimulation.

### DETAILED DESCRIPTION OF THE INVENTION

Referring now to the single FIGURE of the drawing in detail thereof, there is shown embodiments of a system 1 for wireless control of a stimulation device 100, here e.g. for spinal cord stimulation according to the present invention. The invention can also be applied to other electrotherapy devices.

Usually, patients 2 use a component 200 such as a remote control (e.g. in the form of a smartphone) throughout the day in order to adjust proper stimulation parameters for the spinal cord stimulation (SCS) for activities such as sitting (e.g. at a desk), or walking, or driving a car. Also stimulation during the day may be different from stimulation during night time etc. For operating an SCS stimulation device 100 patients may use a component 200 such as a smartphone 200 or an actual remote control when less familiar with a touch screen/smartphone. However, in certain situations handling and operating of such operating means may difficult and cumbersome.

Here, the present invention allows a gesture or movement-based operating of the stimulation device that can be accomplished by the patient in an easy manner.

For this, as shown in the FIGURE, the system 1 contains the stimulation device 100 for delivering SCS having a plurality of electrodes 101. The stimulation device 100 is configured to deliver spinal cord stimulation via the electrodes 101 according to one or several parameters that can be adjusted by the patient.

Particularly, the stimulation device 100 may comprise two flexible carriers 102 for carrying the electrodes 101, the carriers 102 may get tunnelled during implantation to the vicinity of the stimulation device (e.g. an implantable pulse generator or IPG) 100 that is typically implanted subcutaneously in the patient's lower abdominal or gluteal region. The carriers 102 may terminate proximally in connectors that are then inserted into the IPG 100 header to allow conducting electrical charge to the electrodes 101. However, any other stimulation device design may also be used.

Now, according to the present invention, as indicated in the FIGURE, the external control device used in the present invention can be a smartphone 200 used alone or in conjunction with one or several finger rings 300, or a bracelet or smartwatch 400. Further, the external control device may also be formed by one or several finger rings 300 (without the help of a device such as a smartphone 200).

Particularly the external control device 200, 300, 400 of the respective embodiment is configured to transmit a control signal C to the stimulation device 100 that can be received by a corresponding unit 103 of the stimulation device 100 when the external control device detects a change in translational T and/or rotational R motion, or detects a contactless interaction G, e.g. a gesture G, that the patient forms with a hand.

Particularly, in an embodiment indicated in the FIGURE the external control device is formed as the smartphone 200 that provides a control interface to the stimulation device 100 via a radio connection (e.g. Bluetooth) C, wherein the external control device 200 further contains a magnetic field sensor that senses the orientation of the external control device 200 relative to the earth's magnetic field such that when the external control device undergoes a rotation R initiated by the patient when e.g. lying on a surface, a corresponding rotation angle is coded as a control signal C and transmitted to the stimulation device 100 which adjusts a selected parameter of the electrical stimulation accordingly.

Further, in an alternative embodiment shown in the FIGURE, the system 1 may further contain one or several rings 300 configured to be arranged on fingers of the patient 2, respectively, which ring(s) 300 are configured to be connected to the external control device (e.g. smart phone) 200 via a radio connection (e.g. Bluetooth), wherein the respective ring 300 contains a motion sensor. The smartphone 200 again acts as control interface to the stimulation device 100 via a radio connection (e.g. Bluetooth) C such that when the patient performs (e.g. taps) a specific finger pattern or movement that is detected by the ring(s) 300, a corresponding control signal C is transmitted from the smartphone 200 to the stimulation device 100 which adjusts a selected parameter of the electrical stimulation generated by the stimulation device 100 accordingly.

Furthermore, in a further embodiment, the smartphone 200 may also be configured to detect a specific motion or gesture of a patient's hand and/or fingers by use of a camera. Also here the gesture is transformed into a corresponding control signal C that is transmitted to the stimulation device 100 and lead to a corresponding adjustment of a selected parameter of the electrical stimulation applied by the stimulation device 100.

Furthermore, in an alternative embodiment shown in the FIGURE, the external control device can formed as a smartwatch 400 that has a software installed that provides a control interface to the stimulation device 100 via a radio connection (e.g. Bluetooth) C. The smartwatch 400 contains a motion sensor that senses a specific movement of the patient (e.g. of its wrist) that is then translated into a corresponding control signal C which transmitted to the stimulation device 100 which in turn adjustment a selected parameter of the electrical stimulation applied by the stimulation device 100 accordingly.

In a further embodiment, instead of the smartwatch 400, also a bracelet 400 can be used which then contains the motion sensor configured to detect complex finger gestures G or wrist movements.

Furthermore, the external control device can also be formed by one or several pressure-sensitive finger rings 300 that are configured to detect gestures G of the hand of the patient wearing the ring(s) 300. Here, a further device such as a smartphone 200 can be omitted and the ring(s) may be configured to transmit a corresponding control signal C to the corresponding unit 103 of the stimulation device 100 which in turn adjusts the stimulation parameter in question using the control signal C.

For example, the following parameters may be identified by certain gestures G and or movements T, R and particularly adjusted by these gestures G or movements T, R:
a) stimulation amplitude;
b) stimulation pulse width;
c) stimulation frequency and/or stimulation cycles; and
d) electrode or electrodes for stimulation from a plurality of electrodes, e.g. selection of one or more electrodes from an array of electrodes.

Thus, to summarize, the present invention particularly allows the individual patient or caregiver to configure the system 1 to associate gestures/movements with changes in therapy delivery parameters.

In this way the user interaction is reduced from having to push several buttons on the remote control device to simply performing a gesture or certain movement.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. A system utilizing wireless control, the system comprising:
an implantable stimulation device for delivering electrical stimulation to a patient;
an external control device configured for transmitting a control signal to said implantable stimulation device when said external control device detects:
a change in translational and/or rotational motion of said external control device; or
a contactless interaction of the patient with said external control device.

2. The system according to claim 1, wherein:
said implantable stimulation device is configured for spinal cord stimulation; and
said implantable stimulation device has a plurality of electrodes, said implantable stimulation device is configured to deliver spinal cord stimulation via said electrodes.

3. The system according to claim 1, wherein the contactless interaction with said external control device is a motion gesture of the patient in front of said external control device.

4. The system according to claim 1, wherein said external control device is selected from the group consisting of a hand-held device, a smartphone, a smartwatch, an object configured to be worn by the patient, a bracelet and a ring.

5. The system according to claim 1, wherein the electrical stimulation has at least one parameter being adjusted by means of said the control signal, the at least one parameter being selected from the group consisting of a stimulation amplitude, a stimulation pulse width, a stimulation frequency and/or stimulation cycles, and electrode selection for stimulation from a plurality of electrodes.

6. A method for wireless control of an implantable stimulation device using an external control device, which comprises the steps of:
detecting a change in a translational and/or rotational motion of the external control device or a contactless interaction of a patient with the external control device by the external control device; and
transmitting a corresponding control signal by the external control device to the implantable stimulation device for controlling the implantable stimulation device.

7. The method according to claim 6, wherein:
the implantable stimulation device is configured for spinal cord stimulation; and
the implantable stimulation device has a plurality of electrodes, the implantable stimulation device is configured to deliver spinal cord stimulation via the electrodes.

8. The method according to claim 6, wherein the contactless interaction with the external control device is a motion gesture of the patient in front of the external control device.

9. The method according to claim 6, which further comprises selecting the external control device from the group consisting of a hand-held device, a smartphone, a smartwatch, an object configured to be worn by the patient, a bracelet and a ring.

10. The method according to claim 6, which further comprises adjusting one of the following parameters by means of the control signal:
a stimulation amplitude;
a stimulation pulse width;
a stimulation frequency and/or stimulation cycles; and
a selection of at least one electrode for stimulation from a plurality of electrodes.
